(19) United States
(12) Patent Application Publication
(10) Pub. No.: US 2008/0038807 A1
(43) Pub. Date: Feb. 14, 2008

(54) Technical Process And Plant For Extraction And/Or Encapsulation Of Living Cells From Organs

(76) Inventor: Rainer Pommersheim, Mainz (DE)

Correspondence Address: BODNER & O'ROURKE, LLP, 425 BROADHOLLOW ROAD, SUITE 108, MELVILLE, NY, 11747, US

(21) Appl. No.: 10/591,280

(22) PCT Filed: Feb. 23, 2005
(86) PCT No.: PCT/EP05/01893

§ 371 Date: Jun. 22, 2007

(30) Foreign Application Priority Data
Mar. 5, 2004 (DE) 10 2004 011 400.5

(51) Int. Cl. C12N 13/00

(52) U.S. Cl. 435/173.9

(57) ABSTRACT

The invention relates to a method and a corresponding plant for the extraction and/or encapsulation of living cells from organs. In a first step, the organ containing the cells is disintegrated in an enzymatic process into individual cells or cell agglomerations. The relevant cells are then isolated from the obtained cell mixture. The so extracted cells can then be encapsulated. The invention describes a technical process and a plant combining these three steps.

The invention relates to a method and to the corresponding plant for the extraction and/or encapsulation of living cells from organs. In a first step, the organ containing the cells is disintegrated in an enzymatic process into individual cells or cell agglomerations. The relevant cells are then isolated from the obtained cell mixture. The so extracted cells can then be encapsulated. The invention describes a technical process and a plant combining these three steps.

In medical science or pharmacy, but also in the technological practice, it is more and more frequently required to make use of living cells. To improve the handling capability and also the keeping quality thereof they are used in an encapsulated form.

In the development of drugs, for example, the active substances are examined for their effect in the liver. This requires laborious animal experiments and expensive clinical tests. Although hepatic cells are available in large amounts from the meat industry, the development of a test kit on the basis of isolated hepatic cells has failed so far because the individual cells remain alive only for a few hours. By isolating the cells from the liver and by encapsulating them subsequently it is possible to prepare the cells to remain alive for several weeks, so that they can be used, for the first time, for toxicological tests within the scope of standard test kits.

Another approach relates to the therapy of diseases like, for example, the diabetes mellitus by means of transplanting living encapsulated islet cells. The cells are isolated from the organ and encapsulated such that they are protected against the immune system inherent in the body. This allows the transplantation of dissimilar cells. If one encapsulates, for example, porcine islet cells and gives an injection thereof to a patient suffering from diabetes, the cells would not only produce the necessary insulin, but would also control the blood sugar. A large number of such tests are described in the prior art.

In all of the aforementioned approaches the cells have to be extracted, i.e. isolated, from the organ in a first step. So far, two basically different methods have been adopted in the laboratory practice: 1. Chopping up the organ with mechanical means and regenerating the obtained cell and tissue suspension subsequently. 2. An enzymatic disintegration of the organ into individual cells and subsequent isolation of the relevant cells from the mixture.

The U.S. application U.S. Pat. No. 5,079,160, for example, describes a method for extracting living cells from the organs of mammals. This is accomplished by destroying the connective tissue of the organ with an enzyme in a first step, whereby the individual cells are set free. The enzyme is inactivated by means of cooling. The cell suspension is subsequently separated in a density gradient. The patent document also describes a laboratory system for this purpose. In accordance with the method described therein, and with the laboratory system as described, a disintegration of the organs is not possible in technical automated methods. Also, no information are provided with respect to a subsequent encapsulation of cells.

In order to be able to manipulate the cells or cell agglomerations it is common practice to encapsulate them subsequently. To achieve this, they are admixed to a liquid, usually water-soluble basic substance in a first step, which is then transformed into droplets by suitable devices. The formed droplets are hardened and encapsulate the material dissolved or suspended in the same or the cells. As a rule, this is achieved by cross-linkage in a precipitation bath or by changing physical parameters. The spherules so formed, the diameter of which ranges from some micrometers to some millimeters, may be coated in a next step.

In the prior art methods are described in several places, which relate to an encapsulation of living cells. For example, G. Troost et al. (G. Troost et al. champagne, sparkling wine, Stuttgart 1995) describes yeast immobilized in alginate spheres for the bottle fermentation in the production of sparkling wines. By this, the time-consuming manual riddling off the yeast depot can be replaced by the fast sedimentation of the spherules in the champagne bottle. Any extraction of cells from organs is not described because it is not necessary.

F. Lim and A. Sun describe in the magazine “Science”, volume 210, pages 908-910, 1980, a capsule having a semi-permeable membrane for the immobilization of living cells, whereby the core of the capsule is surrounded by a single layer of an Ply-l-Lysin/alginate complex. With these capsules, the cells are prevented from escaping from the core of the capsule. However, this membrane capsule is not suited for the use in technical processes owing to its relatively small mechanical stability. Also, it is impossible to encapsulate therein molecules having the size of an enzyme or smaller, as the membrane is permeable with respect to the same. This method also is the subject matter of the U.S. application U.S. Pat. No. 4,323,457. In the embodiment as described it is not suitable for a technical process and also does not deal with the extraction of cells.

The patent application DE 43 12 970.6 describes a membrane capsule which is also suited for the immobilization of enzymes and proteins, but also of living cells. The core containing the immobilized material is surrounded by a multi-layer envelope, with each of these layers imparting a certain property to the entire envelope. By selecting the envelope polymers in an advantageous manner the permeability of the membrane can be reduced such that also enzymes remain in the capsule, while the much smaller substrates and products can pass through the membrane. These capsules can so far only be produced on a laboratory scale, however, i.e. in small amounts. Here, too, there is no indication to a method for the extraction of cells.

All of these methods always relate to one step of the process only, i.e. either to the extraction of cells or to the encapsulation, or they are only suited for laboratory sizes, i.e. not for technical processes.

On the basis of this prior art it is the object of the invention to provide a method and an associated plant allowing, for the first time, to extract, separate and encapsulate living cells from an organ in a technical process.

The production process according to the invention is classified into three phases, the cell extraction, the cell separation and the cell encapsulation.

The organ from which the cells are extracted is disintegrated into individual cells in a first step. This is accomplished with an enzymatic process, the principle of which is known from the prior art. In a second process step, the cell suspension obtained is separated, whereby the cell type relevant for the further processing is separated from the mixture by means of an antibody marker. If an encapsulation of the obtained cells is necessary, this may be achieved in a next process step. The encapsulation is based on the principle according to which the relevant cells are, in a first step, admixed to a liquid, usually water-soluble basic substance, from which mechanically stable, coatable particles are obtained by transforming it into droplets and hardening the same.

A machine on which such a process is based therefore consists of three modules, one for each process step: cell extraction, cell separation and cell encapsulation.

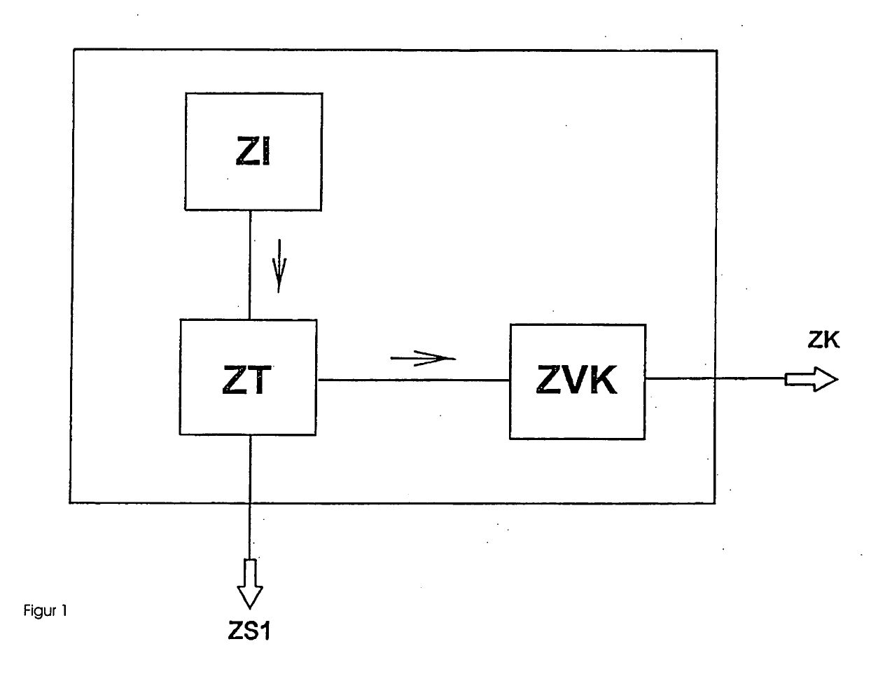

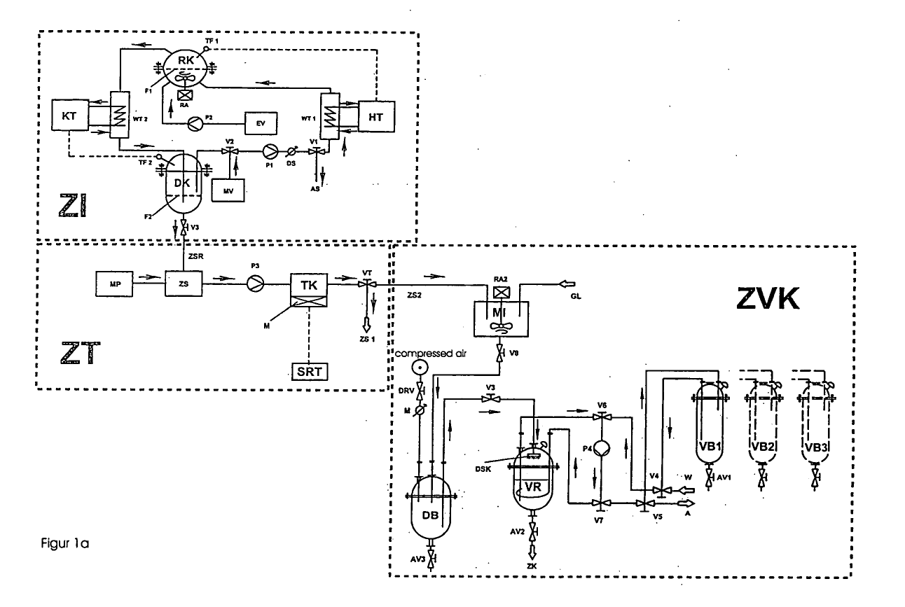

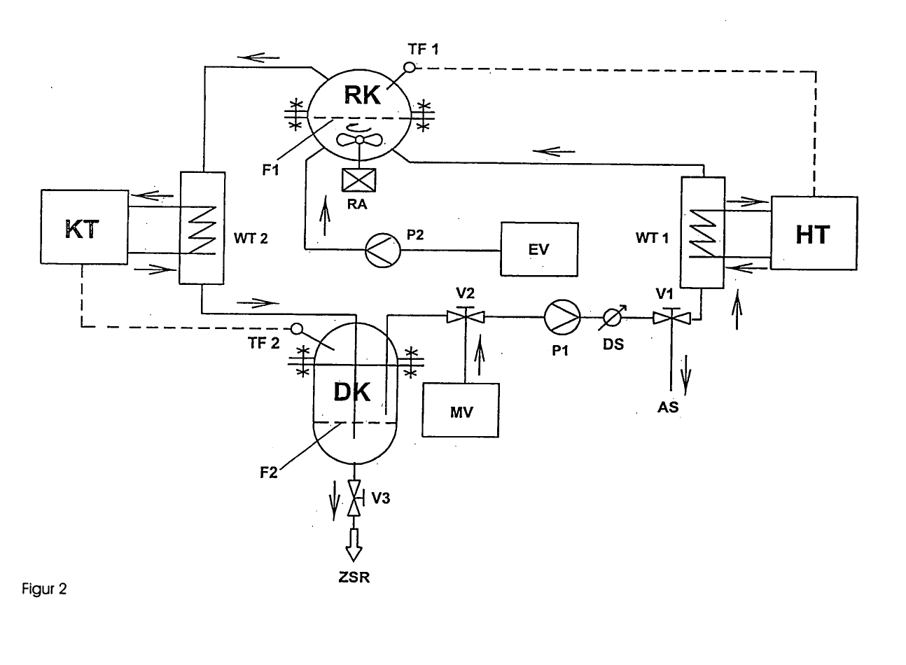

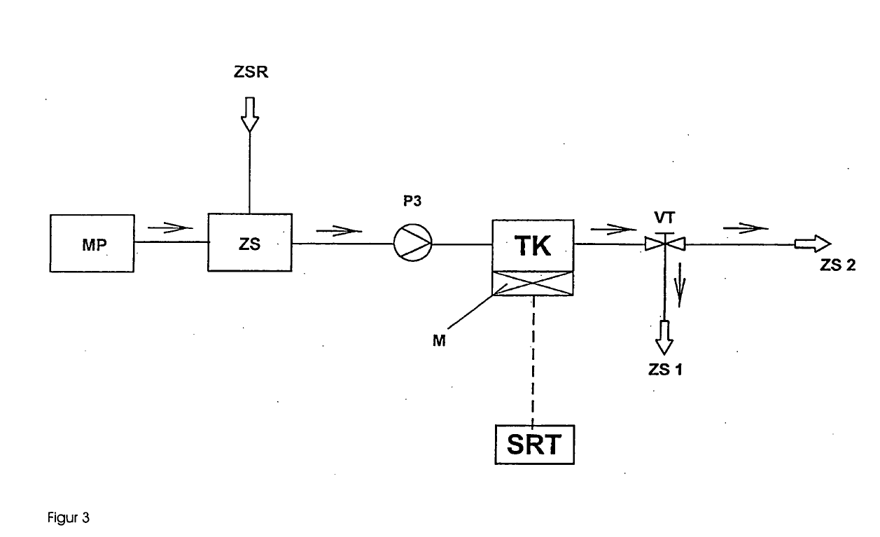

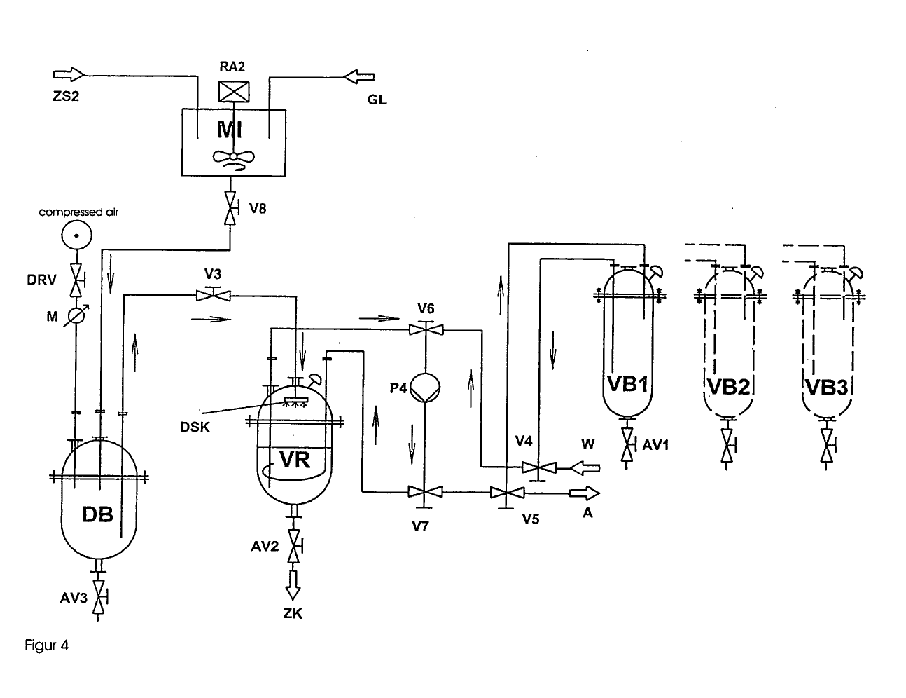

FIG. 1 and FIG. 1a show the basic structure of a plant in which the method according to the invention has been implemented. All components of the machine are fabricated such that the plant can be sterilized by autoclaving. The cell extraction is accomplished by a disintegration of the organ into individual cells and/or cell agglomerations. This takes place in module ZI. The exact structure and operating mode of the cell isolation module (ZI) is illustrated in FIG. 2 and will be explained in more detail below. After the isolation the cell mixture is transferred into the cell separation module ZT. The structure of the module for separating the cells ZT is schematically illustrated in FIG. 3. The operating mode thereof will be described below. A subsequent encapsulation of the relevant cells can be performed by means of module ZVK. The structure of this module is illustrated in FIG. 4, and the operating mode thereof will be explained in one of the following paragraphs.

FIG. 2 schematically illustrates the cell isolation module (ZI) of the plant. The operation mode thereof is as follows: The organ of a recently deceased, e.g. animal donor is placed on the perforated plate F1 in the reaction chamber RK. Next, an enzymatic solution is supplied to the organ from the reservoir EV via the metering pump (e.g. a piston pump) P2. Such an enzyme can be, for example, a collagenase. The machine is constructed such that the reaction chamber can be removed, so that the organ can be placed into the chamber under sterile conditions and, if required, the enzymatic solution can be fed directly into a blood vessel of the organ through a feed line. The reaction chamber RK forms part of a closed cycle in which it is flushed with a cell culture medium during the whole cell isolation process. This medium is heated from the reservoir MV via the pump P1 and via the valves V2 and V1 in the heat exchanger WT1 to approximately 35-38° C. and is passed into the chamber RK. P1 can be, for example, a gear pump or another self-priming pump with a detachable pump head. The pump head can thus be autoclaved together with the rest of the machine. The heat exchanger WT1 is connected to a heating thermostat HT, which detects with the temperature sensor TF1 the temperature in the chamber RK and controls it to a temperature of approximately 35-38° C. At this temperature the enzyme, the collagenase, is active and disintegrates the connective tissue of the organ so that the individual cells are extracted and set free. To support this process a turbulent mixing of the culture medium is produced inside the chamber RK by means of a stirrer RA.

The cells that have been set free are captured by the culture medium flowing through the chamber RK and are passed via the heat exchanger WT2 into the decantation chamber DK. In this process the culture medium including the cells are cooled to approximately 3-8° C. so that the enzyme, the collagenase, is inactivated. The temperature is controlled by a cooling thermostat KT. The thermostat KT is connected to the temperature sensor TF2, which constantly detects the temperature in the decantation chamber DK, and controls it to approximately 3-8° C. The inlet pipe for the culture medium (including the cells) is passed into the interior of the decantation chamber DK through the filter frit F2. This filter frit is made, for example, of special steel and has a porosity smaller than the diameter of the cells isolated from the organ (e.g. 5 μm). In this way, the cells are separated from the culture medium and collected underneath the frit. The frit is permeable with respect to the culture medium. The latter is pumped off again above the frit and is returned to the cycle by a corresponding position of the valve V2 and V1. The cycle also comprises a pressure switch DS which correspondingly controls the pump P1 if the filter frit F2 is clogged and an excessive pressure increase occurs in the system. By opening the valve V3 the isolated cells are passed as cell suspension ZSR out of the decantation chamber and can be supplied to the cell separation module ZT. If the plant is to be cleaned, the corresponding rinsing solution is sucked in via valve V2 and pumped through the system. After having passed therethrough the rinsing solution can be removed from the cycle by opening V1.

The suspension ZSR obtained by the cell isolation is a mixture of different cell types. In some applications the suspension may be used in this form. As a rule, however, a specific cell type has to be separated from the mixture. Methods for separating cell mixtures are described in the prior art at several places. Apart from the classical separating method in a density gradient, followed by a centrifugation of the individual fractions, the separation with magnetically marked antibodies is increasingly implemented. In this method specific antibodies are used, which contain magnetic particles. These antibodies settle on certain cell types and render them magnetic, which allows their separation out of the cell mixture in a magnetic field. If all cells but one specific cell type are marked one talks about a negative marking. In the reverse case, in which only one specific cell type is marked, a positive marking is concerned.

For the separation of the suspension obtained in module ZI the present invention uses the method with specific magnetic antibodies. This process step is technically implemented in module ZT. The structure of this module is schematically illustrated in FIG. 3.

The cell separation module according to FIG. 3 operates as follows: The raw suspension ZSR from ZI is collected in a container ZS where the magnetically marked antibody from MP is metered. Depending on the further use of the cells this antibody can either effect a positive or a negative marking. As example the further description is based on a negative marking. The so marked cell mixture is pumped through pump P3 into the separation chamber TK. P3 is, for example, a hose pump or any other pump suitable for pumping cell suspensions due to their design. The separation chamber comprises channels through which the suspension is passed.

Below the chamber a magnet M is disposed. If this magnet is a permanent magnet, the chamber has a mechanism allowing for the removal of the magnet (SRT). If the magnet is an electromagnet, it comprises a control mechanism (SRT) by means of which it can be activated or deactivated. In the chamber, the marked cell suspension is exposed to a magnetic field so as to retain the marked cells. In the case of a negative marking only the cells relevant for the further processing are transported by the liquid via VT. One obtains a homogeneous cell suspension ZS2 in the cell culture medium. By removing the magnetic field also the marked cells are now transported further by the liquid and flushed out as cell suspension ZS1 by switching the valve VT.

The obtained cells may be used directly as suspension ZS1 or ZS2. With quite a number of cells it is advantageous, however, to encapsulate them in an additional step. Thus, the durability of the cells can be increased and their handling can be improved.

FIG. 4 schematically shows the cell encapsulation module ZVK of the process. It allows an encapsulation of the cells both in so-called membrane capsules, but also in membrane-free capsules. In a mixing vessel Ml equipped with a stirrer RA2 the cell suspension ZS2 is suspended or dissolved in a base material solution GL, preferably sodium alginate. This base material suspension or solution is then transported via V8 into the pressure vessel DB, and from there via V3 into the encapsulation reactor VR. This can either be accomplished with compressed air, as shown in FIG. 3 (control by valve DRV and manometer M), or pumps, screw conveyors etc. may be used. Then, by instilling this suspension or solution into a precipitation bath by means of the nozzle head DSK spherules are formed. This can either be effected by the complex formation with a polyvalent saline solution, e.g. if alginate is used, or by changing the physical parameters, e.g. the temperature, if other base materials are used. For transforming the liquid into droplets several methods may be applied, depending on the desired size, productivity and size distribution. To this end, either nozzles having capillaries can be used at which the droplet is separated by an air flow, or those at which the droplet separation is achieved with vibration, electrostatic deflection etc.

When immersing the liquid droplet in the precipitation bath it turns to gel and encloses the material to be encapsulated. Prior to the start of the instillation process the required precipitating reagent is conveyed from the reservoir VB1 into the encapsulation reactor via valves V4, V6, V7 with the aid of pump P4. Due to the tangential introduction of the liquid no additional stirring is necessary. During the production of the droplets the precipitating reagent is carried in the cycle due to a suitable position of valves V6 and V7 and by means of pump P4. Once the droplet production is completed and the particles are hardened the precipitating reagent is pumped back into the container VB1 via valves V6, V7 and V5. If the reagent is exhausted, it may also be discarded by a corresponding position of V5. Next, a wash solution is pumped into the reactor VR via valves V4, V6 and V7, so that the spherules are freed from the excess precipitating agent, i.e. washed.

If a coating of the spherules is desired, the corresponding coating solutions can—in a similar process—be pumped from the reservoirs VB2, VB3 etc. into the reactor VR, and can again be removed from the same. The coating of the gel particles is accomplished by contacting them with the respective coating solutions. These are diluted aqueous solutions of polymers with anionic or respectively cationic groups, such as chitosan, polyvinyl pyrrolydone, polyethylene imine, carboxymethyl cellulose, alginate, polyacrylic acid etc., which form so-called polyelectrolyte complex layers on the surface of the capsule. By repeatedly immersing the particles in these solutions, as is described in P 43 12 970.6, several layers of the capsule envelope are formed.

Via valve AV2 the encapsulated cells are flushed out of the reactor VR as suspension ZK. Depending on the field of application at a later time the capsules may afterwards either be incubated, frozen or dried.

1. Method and plant for the extraction and/or encapsulation of living cells from organs, characterized in that the organ containing the cells is disintegrated in an enzymatic process into individual cells and/or cell agglomerations, that the relevant cells are subsequently separated from the cell mixture thus obtained and can then be encapsulated.

2. Method according to claim 1, characterized in that it comprises some or all of the following steps, which can also be repeated several times:
flowing a nutrient fluid heated to approximately 35-38° C. around an organ
extracting cells from the organ by means of an enzyme
transferring the extracted cells through the nutrient fluid in form of a suspension
cooling the cell suspension thus obtained to approximately 3-8° C.
concentrating the cell suspension by separating the cells from the suspension with a porous frit
after the separation of the cells, returning the nutrient fluid into a cycle
marking specific cell types in the concentrated suspension by means of magnetically marked antibodies
separating the so marked cells from the suspension in a magnetic field
suspending the relevant cell fraction in a base material
transforming this base material suspension into droplets
precipitating the droplets
rinsing and suspending the spherules formed by the precipitation in a washing liquid
flowing a polycationic polymer solution around the spherules and forming a cationic charge on the surface of the spherules
washing the spherules with a washing liquid
washing the spherules with a detergent solution
flowing a polyanionic polymer solution around the spherules and forming an anionic charge on the surface of the spherules
rinsing and suspending the spherules formed by the precipitation in a washing liquid
suspending the spherules formed by the precipitation with the cells in a cell culture
incubating the spherules with the cells
freezing the spherules with the cells
drying the spherules with the cells.

3. Method according to claim 2, characterized in that the enzyme used for the cell isolation is a collagenase.

4. Method according to claim 2, characterized in that the base material into which the cells are stirred for the encapsulation is a soluble natural material or synthetic material.

5. Method according to claim 2, characterized in that the base material is transported into a device for producing droplets by mechanical means, preferably a screw conveyor or a pump.

6. Method according to claim 2, characterized in that the base material is transported pneumatically into a device for producing droplets.

7. Method according to claim 2, characterized in that the device for producing droplets forms part of a reaction vessel.

8. Method according to claim 2, characterized in that the base material is transformed into droplets by vibration, an air flow, a rotational movement (centrifugal forces) and/or by emulsification.

9. Method according to claim 2, characterized in that the produced droplets can be precipitated chemically, e.g. by the influence of salts.

10. Method according to claim 2, characterized in that the produced droplets can be precipitated physically, e.g. by a temperature change.

11. Method according to claim 2, characterized in that the precipitated droplets contain the living cells extracted from an organ.

12. Method according to claim 2, characterized in that the precipitated droplets are kept suspended in the precipitating bath.

13. Method according to claim 2, characterized in that the precipitated droplets are kept suspended in the precipitating bath by stirring.

14. Method according to claim 2, characterized in that the precipitated droplets are kept suspended in the precipitation bath by the flow rate of the surrounding medium.

15. Method according to claim 2, characterized in that the precipitated droplets are coated by flowing suitable polymer solutions around them.

16. Method according to claim 2, characterized in that the precipitated droplets are kept suspended during the coating.

17. Method according to claim 2, characterized in that the precipitated droplets are kept suspended during the coating by stirring.

18. Method according to claim 2, characterized in that the precipitated droplets are kept suspended during the coating by the flow rate of the surrounding medium.

19. Method according to claim 2, characterized in that the coated spherules have an envelope fully enclosing the core and thus the encapsulated material.

20. Method according to claim 2, characterized in that the envelope of the coated spherules is formed of one or more radially arranged layers.

21. Method according to claim 2, characterized in that the layers of the envelope may be portions of different density.

22. Method according to claim 2, characterized in that the coated spherules can be stored and used in an undried, i.e. moist condition.

23. Method according to claim 2, characterized in that the coated spherules are can be freeze-dried.

24. Method according to claim 2, characterized in that the coated spherules can be air-dried.

25. Method according to claim 2, characterized in that solutions applied for precipitation and/or coating are used either as concentrates or ready for use in a diluted form.

26. Plant according to claim 1, which operates according to a method according to claim 1, characterized in that it comprises some of the following main components:
reaction chamber for receiving the organ, comprising a perforated plate and a stirrer (RK)
cooling (KT) and heating (HT) thermostat
heat exchanger for controlling the temperature of the liquids (WT1, WT2)
decantation vessel with porous frit and tubular feedthrough (DK)
chamber for separating marked mixtures in the magnetic field (TK)
mixing container for the base material and the cells (MI)
reservoir for the precipitation bath (VB1)
reservoir for the coating solutions (VB2, VB3, etc.)
reaction vessel for transforming the base material cell suspension into droplets and precipitating the same (VR)
device for drying the coated spherules
pumps (P1, P2, P3) and valves (V1, V2, . . . )
corresponding control components

27. Plant according to claim 26, characterized in that it operates in accordance with FIG. 1 and respectively FIG. 1a and/or that its components are arranged and/or connected to each other in accordance with FIG. 1 and respectively FIG. 1a.

28. Plant according to claim 26, characterized in that it comprises a cell isolation module operating in accordance with FIG. 2 and/or that the components thereof are arranged and/or connected to each other in accordance with FIG. 2.

29. Plant according to claim 26, characterized in that it comprises a cell separation module operating in accordance with FIG. 3 and/or that the components thereof are arranged and/or connected to each other in accordance with FIG. 3.

30. Plant according to claim 26, characterized in that it comprises a cell encapsulation module operating in accordance with FIG. 4 and/or that the components thereof are arranged and/or connected to each other in accordance with FIG. 4.
